(19) United States
(12) Patent Application Publication
(10) Pub. No.: US 2009/0088770 A1
(43) Pub. Date: Apr. 2, 2009

(54) ANGLED SURGICAL DRIVERS AND METHODS OF USE

(75) Inventor: Roy K. Lim, Germantown, TN (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 11/865,116

(22) Filed: Oct. 1, 2007

(51) Int. Cl. A61B 17/58

(52) U.S. Cl. 606/104

(57) ABSTRACT

The present application is directed to a driver for use in surgical procedures. The driver includes a shaft with an elongated shape for accessing locations within a patient. The shaft includes a first section and a second section that may be positioned at various relative angles depending upon the context of use. A mount is positioned at an end of the shaft to receive a variety of tool bits. A drive mechanism extends through the shaft to provide rotation to the mount and engaged tools. The drive mechanism includes a first wheel in the first section of the shaft and a second wheel in the second section. The wheels include rounded nubs that mesh together to transfer the rotational torque from the first section to the second section when the sections are at the various relative angles.

### BACKGROUND

The present application is directed to a surgical driver and, more particularly, to a surgical driver with first and second shafts that may be placed at a variety of angular positions.

Many surgical procedures require a driver tool to access small, tight locations within a patient. The locations may be associated with the spine, lower extremities, internal cavity, and various other areas within the patient. Drivers have historically included a shaft with a handle on a first end and a tool on a second end. The shaft and tool are often coaxially aligned thus forming an overall straight shape for the driver. This shape is effective for accessing some locations, but often times it is necessary to access a location where the tool is offset at an angle relative to the shaft.

Many existing drivers are also cumbersome for use by the surgeon. This may include an overall size of the driver being too large thereby making it difficult to manipulate. Further, the overall size may prevent the driver from being able to access small locations within the patient.

Existing drivers may also include a complicated design with various small components that interact together during use of the driver. These components may become clogged with biological material during the procedure. Further, the small sizes may cause the components to jam or otherwise lock together during use thus rendering the driver useless. These components should be designed to prevent clogging by biological material, and ensure that they remain operational during use.

### SUMMARY

The present application is directed to drivers for use in surgical procedures. The drivers may include a hollow shaft with a distal section and a proximal section. A mount may be attached to an end of the distal section. The mount may include an adjustable opening configured to receive a variety of different tool bits. A drive mechanism may extend within an interior of the shaft. The drive mechanism may include a drive input at an end of the proximal section and a connection in the distal section to connect to the mount. The drive mechanism may also include a connection mechanism to position the distal section at a non-coaxial orientation with the proximal section. The connection mechanism may include a first and second wheels each with rounded nubs that may mesh together to transfer the rotational torque from the proximal section to the distal section and mount.

### BRIEF DESCRIPTION OF THE DRAWINGS

FIG. 1 is a side view of a driver according to one embodiment.

FIG. 2 is an exploded perspective view of a driver according to one embodiment.

FIG. 3 is a partial side view of a first section and a second section of a shaft according to one embodiment.

FIG. 4 is a partial side view of a first section and a second section of a shaft according to one embodiment.

FIG. 5 is an exploded perspective view of a mount and a plurality of tools that may be used with the driver according to one embodiment.

FIG. 6 is an exploded perspective view of a mount according to one embodiment.

FIG. 7 is a side perspective view of a first wheel engaging with a second wheel according to one embodiment.

FIG. 8 is a bottom perspective view of a first wheel engaging with a second wheel according to one embodiment.

FIG. 9 is a partial side view of a first wheel with nubs according to one embodiment.

FIG. 10 is a side view of a second shaft section pivotally connected to the first shaft section according to one embodiment.

### DETAILED DESCRIPTION

The present application is directed to a driver for use in surgical procedures. The driver includes a shaft with an elongated shape for accessing locations within a patient. The shaft includes a first section and a second section that may be positioned at various relative angles depending upon the context of use. A mount is positioned at an end of the shaft to engage with various tools. A drive mechanism extends through the shaft to provide rotation to the mount and engaged tools. The drive mechanism includes a first wheel in the first section of the shaft and a second wheel in the second section. The wheels include rounded nubs that mesh together to transfer the rotational torque from the first section to the second section when the sections are at the various relative angles.

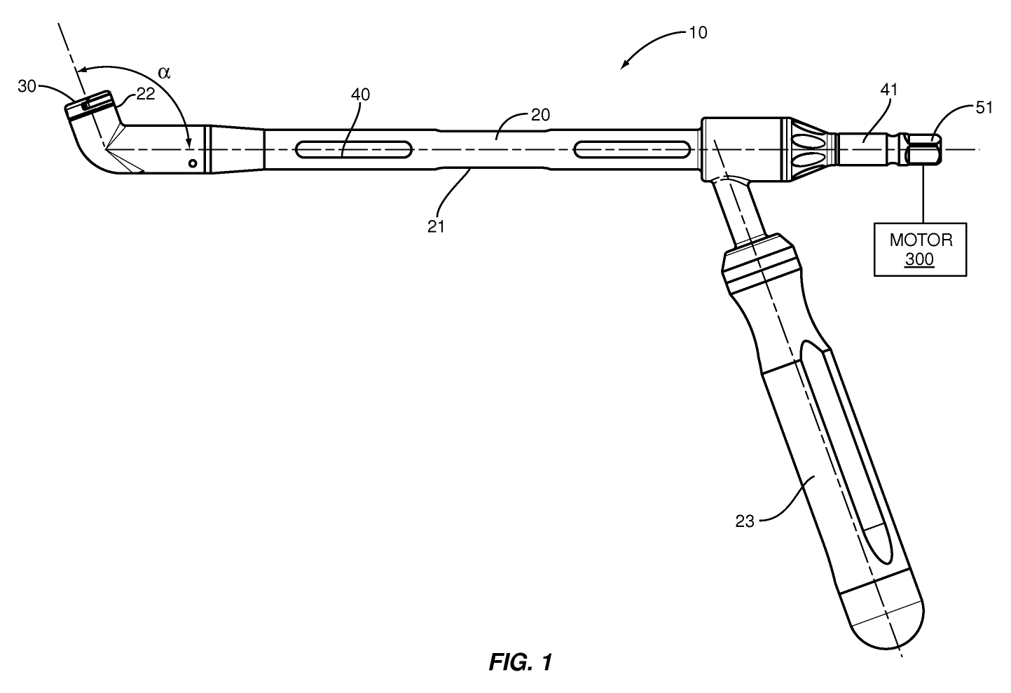

FIG. 1 illustrates one embodiment of the driver 10 that generally includes a shaft 20, a mount 30, and a drive mechanism 40. The shaft 20 includes a first section 21 and a second section 22. The first section 21 is positioned at an angle α relative to the second section 22. The mount 30 is positioned at an end of the second section 22. Mount 30 is configured to receive a variety of different tool bits. The drive mechanism 40 extends through the interior of the shaft 20 and is operatively connected to the mount 30. A drive input 41 is positioned at an end of the shaft 20 to receive a driving force.

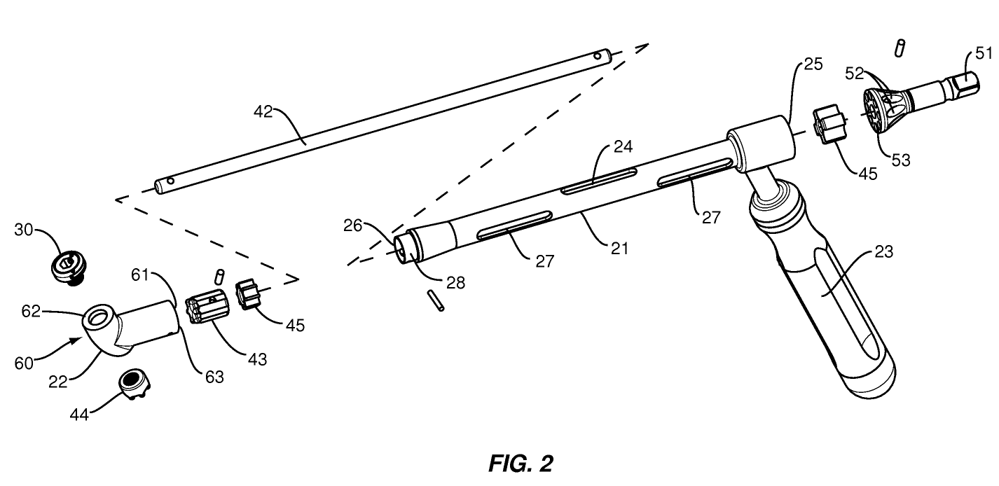

As illustrated in FIGS. 1 and 2, the first section 21 is a hollow tube that extends between a proximal end 25 and a distal end 26. The length and width of the first section 21 may vary depending upon the context of use. FIG. 2 illustrates the first section 21 with a circular sectional shape, although other shapes are also contemplated. An interior space 24 is sized to receive a section of the drive mechanism 40 as will be explained in detail below. A handle 23 may extend outward from the first section 21. Handle 23 is shaped and sized to facilitate grasping by a surgeon. FIGS. 1 and 2 illustrate the handle 23 in proximity to the proximal end 25, although the handle 23 may be positioned at other locations along the first section 21. One or more apertures 27 may be positioned along the first section 21.

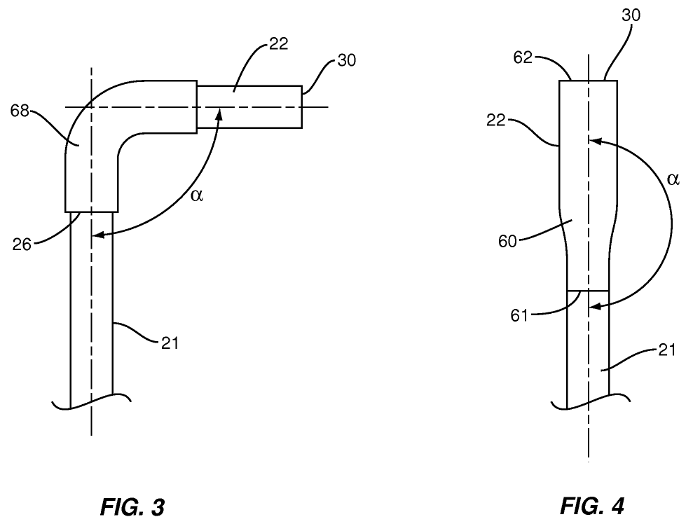

The second section 22 extends from the distal end 26 of the first section 21. Second section 22 is hollow and includes an interior space to receive the drive mechanism 40. In one embodiment as illustrated in FIGS. 1 and 2, the length of the second section 22 is considerably shorter than the first section 21. FIG. 3 illustrates another embodiment with a longer second section 22.

The second section 22 may be positioned at various angles α relative to the first section 21. Angle α is formed between centerlines of the first and second sections 21, 22. FIG. 1 illustrates an embodiment with the angle α being about 110°. FIG. 3 illustrates another embodiment with the angle α being about 90°. In another embodiment as illustrated in FIG. 4, the sections 21, 22 are substantially coaxial with the angle α being about 180°.

In one embodiment as illustrated in FIGS. 2 and 4, a distal section of the first section 21 and the second section 22 are formed by a case 60. Case 60 is hollow and includes a first end 61 and a second end 62. The first end 61 attaches to the distal end 26 of the first section 21. The distal end 26 may include a neck 28 with a reduced width that fits within the interior of the first end 61. Outer surfaces of the first section 21 and the case 60 may be substantially the same to provide a smooth transition which facilitates insertion of the driver 10 within the patient.

A locking mechanism 63 may be associated with the first end 61 and/or the distal end 26 to secure the case 60 to the first section 21. The locking mechanism 63 may include but is not limited to a ball and detent connection, a tab and slot arrangement that provides for inserting the first end 61 onto the distal end 26 and then rotating the case 60 for secure engagement, or threads on each of the first end 61 and distal end 26.

Driver 10 may include a number of different cases 60 depending upon the specific context. The different cases 60 may each include a different angle α to allow the mount 30 and attached tool bits to access the specific location within the patient. The surgeon may determine the desired angle α and then attach the appropriate case 60.

FIG. 3 illustrates another embodiment with a connector 68 positioned between the first and second sections 21, 22. The connector 68 includes a first end that attaches to the distal end 26 of the first section 21 and a second end that attaches to the second section 22. Connector 68 may include different shapes for positioning the sections 21, 22 at various angular orientations.

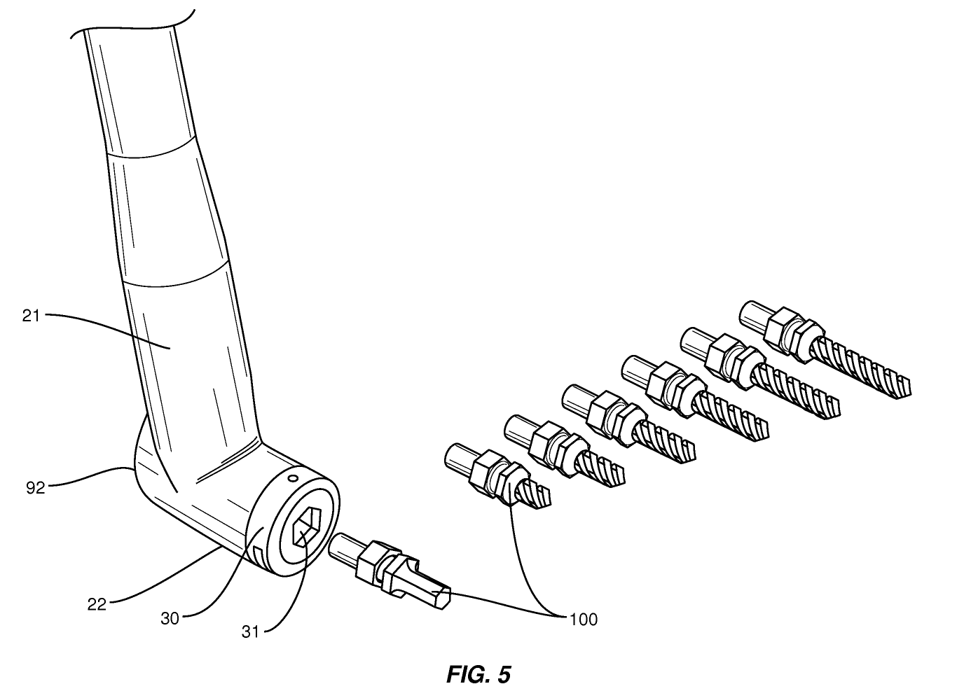

The mount 30 is positioned at the distal end of the second section 22 and provides for engagement with various tool bits 100 as illustrated in FIG. 5. Mount 30 includes an opening 31 sized to receive the various tools 100. Tool bits 100 fit within the opening 31, and may include various drive ends including hexagonal, Torx, Philips, flathead, in addition to various sizes of drills.

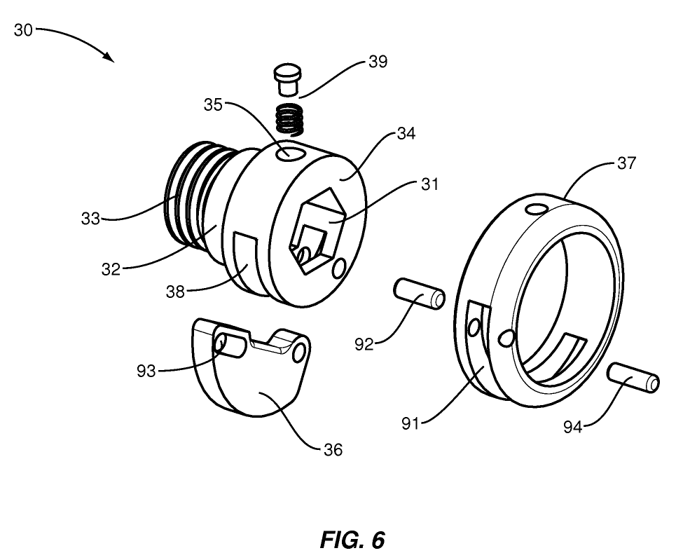

FIG. 6 illustrates an exploded view of one embodiment of the mount 30. Mount includes a collet 32 with a first end 33 that fits within the second section 22, and a second end 34 that faces outward away from the second section 22. The opening 31 extends into the second end 34 to receive the tool bits 100. Collet 32 further includes a slot 38 positioned towards the second end 32 that is sized to receive a clip 36. An aperture 35 in the collet 32 is sized to receive a biasing member and pin 39. A locking ring 37 is sized to fit over the second end 34 and includes a central opening that aligns with the opening 31. Locking ring 37 further includes a slot 91 that aligns with the slot 38 on the collet 32.

In an assembled state, the biasing member and pin 39 are inserted into the aperture 35 and are held in position by the locking ring 37. The clip 36 is inserted through the slot 91 in the locking ring 37 and into the slot 38 in the collet 32. A first pin 94 is inserted through an opening in the collet second end 34 to maintain the clip 36 connected to the collet 32. A second pin 92 is inserted into the through the locking ring 37 and an elongated opening 93 in the clip 36.

In use, the locking ring 37 may rotate in both directions around the collet 32. During rotation, the biasing member and pin 39 contact against an inner surface of the locking ring 37 and provide audible clicks as the pin aligns with apertures in the locking ring 37. As the locking ring 37 rotates, the elongated opening 93 in the clip 36 slides along the pin 92 moving the clip radially inward and outward relative to the opening 31 between open and closed positions. In the open position, clip 36 is slid radially outward away from the opening 31 to allow insertion of a tool bit 100. In the closed position, clip 36 is slid radially inward towards the opening 31 to lock the tool bit 100.

The drive mechanism 40 provides a driving force to rotate the mount 30 and the attached tool bit 100. In the embodiment of FIG. 2, the drive mechanism 40 includes the drive input 41, a drive shaft 42, a first wheel 43, and a second wheel 44.

The drive input 41 is positioned at the proximal end 25 of the shaft 20. Drive input 41 includes a fitting 51 that is operatively connected with a power source 300. Fitting 51 may include a hexagonal head with flat engagement surfaces that operatively connect to the power source 300. Fitting 51 may also include other shapes to engage with the power source 300 to provide a rotational force. A second end of the drive input 41 connects with the drive shaft 42. Second end may include an opening 53 sized to receive the drive shaft. A bushing 45 may extend between the drive input 41 and drive shaft 42 to aid the connection. In one embodiment as illustrated in FIG. 1, the second end of the drive input 41 abuts against the proximal end of the shaft 20.

The drive shaft 42 is an elongated member that extends through the interior 24 of the shaft first section 21. The width of the drive shaft 42 is smaller than the width of the interior 24 to allow the drive shaft 42 to rotate. In one embodiment, the drive shaft 42 includes a circular cross-sectional shape.

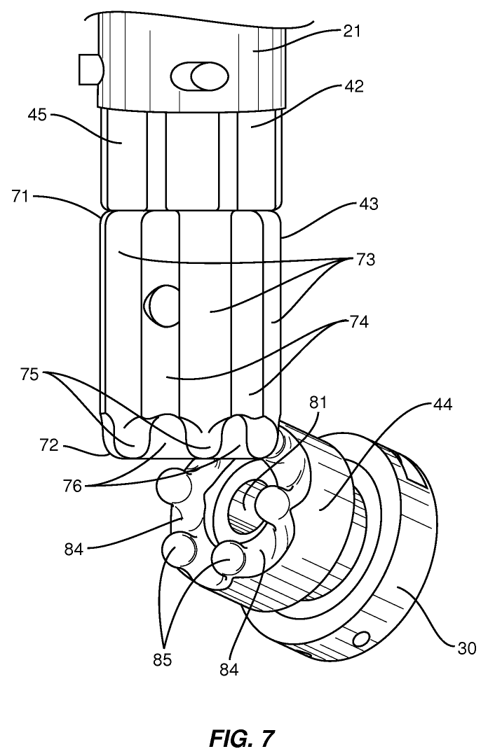

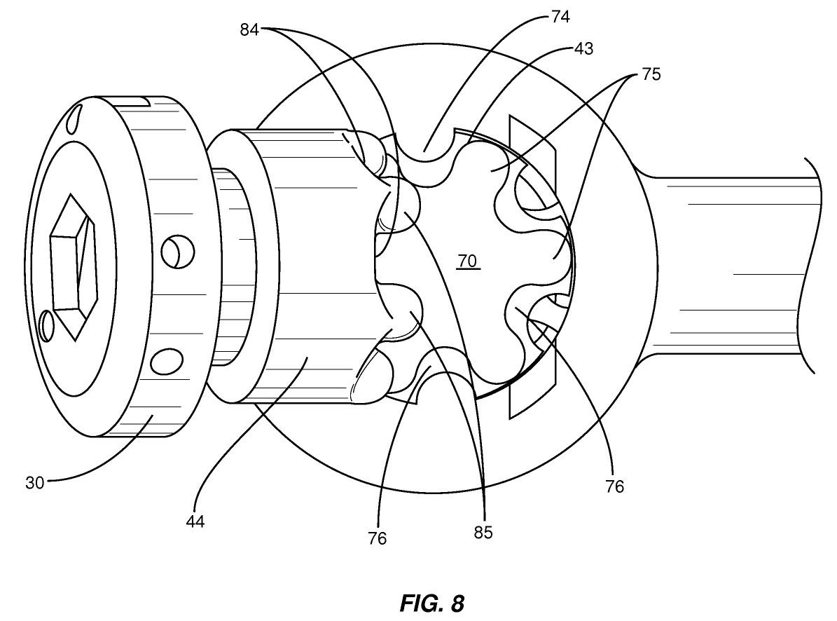

The first wheel 43 is connected to the distal end of the drive shaft 42. In one embodiment, a bushing 45 provides the connection. The details of the first wheel 43 are best illustrated in FIGS. 7 and 8 and include a first end 71 and a second end 72. In one embodiment, the first wheel 43 is substantially cylindrical with the first end 71 in contact with the drive shaft 42 and/or bushing 45. The second end 72 engages with the second wheel 44. In one embodiment, ridges 73 and valleys 74 extend axially along the length of the first wheel 43 between the first and second ends 71, 72. The ridges 73 and valleys 74 are interposed around the circumference of the first wheel 43.

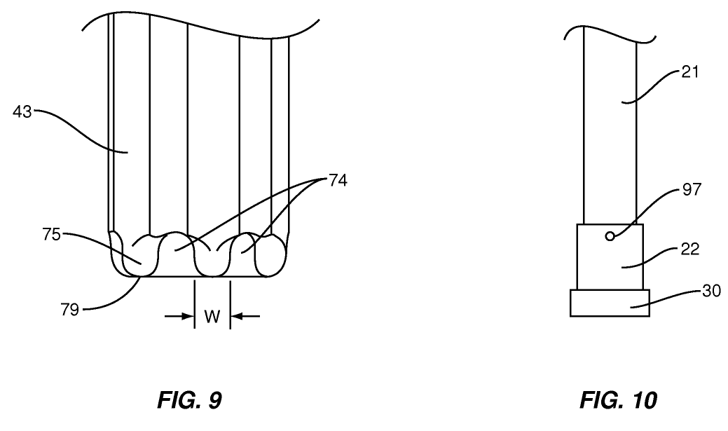

The second end 72 includes a gear member 70 that includes a plurality of nubs 75. In one embodiment, the nubs 75 are axially aligned at the ends of the ridges 73. The nubs 75 include a curved surface that faces axially and laterally outward from the second end 72. In one embodiment as illustrated in FIG. 9, nubs 75 include a substantially constant width w that rounds at the tip 79. In another embodiment (not illustrated), nubs 75 include a narrower mid-section that enlarges towards the tip 79. The valleys 74 may include curved surfaces that connect between each nub 75. The nubs 75 and valleys 74 together give the gear member 70 a prong-like pattern for engaging with the second wheel 44.

The second wheel 44 includes a pattern of nubs 85 and valleys 84 that engage with the first wheel 43. Nubs 85 include rounded surfaces that face axially outward and laterally similar to nubs 75. Nubs 85 are sized to fit within the valleys 74 and contact against the nubs 75 on the first wheel. As best illustrated in FIG. 7, the nubs 85 on the second wheel 44 are each independent members that extend axially outward from the second wheel 44. The nubs 85 extends around the periphery of the second wheel 44 and are each spaced apart by a valley from neighboring nubs 85. A central area 81 at the end of the second wheel 44 is substantially open. The second wheel 44 also includes a second end that attaches to the mount 30.

The rounded surfaces of the nubs 75, 85 and valleys 74, 84 allow the first and second wheels 43, 44 to engage together at a variety of angles. In the embodiment of FIGS. 7 and 8, the first and second sections 21, 22 are positioned at a non-coaxial orientation. This type of orientation causes the wheels 43, 44 to be aligned with contact being limited to a small number of nubs 75, 85 (i.e., less than all the nubs 75, 85). When the sections 21, 22 are in closer axial alignment, more of the nubs 75, 85 engage together. In a co-axial orientation as illustrated in FIG. 4, each of the nubs 75, 85 engage together.

The driver 10 is designed for use in a variety of surgical procedures. In use, the surgeon may initially determine the desired angle for positioning the tool bit 200 within the patient. Once determined, the surgeon positions the second section 22 at the appropriate angle α relative to the first section 21. In one embodiment, this includes selecting and attaching the appropriate case 60 and attaching it to the distal end 26 of the first section 21. Once complete, the desired tool bit 100 is connected to the mount 30.

Next, the power source 300 is operatively connected to the drive input 41. This may include connecting leads from the power source 300 onto the fitting 51 of the drive input 41. The driver 10 is then manipulated by the surgeon to place the tool bit 100 within the patient. The power source 300 is then activated causing the drive input 41 to rotate which in turn rotates the drive shaft 42 and first wheel 43. The nubs 75, 85 engage causing the rotation to be transferred from the first wheel 43 to the second wheel 44. The torque is then transferred to the mount and ultimately to the tool bit 100.

The same angle α be maintained throughout the procedure. Alternatively, it may be necessary for the angle α to vary. In this instances, the driver 10 is removed from the patient and the angle α is adjusted as necessary. Likewise, the surgeon may change the tool bit 100 by removing the driver 10 from the patient, and unlocking the mount 30, replacing the tool bit 100, and relocking the mount.

Driver 10 may also include features to facilitate sterilization. Vents 52 positioned in the drive input 41 and apertures 27 located on the shaft 20 provide for entry and exit points for steam and water. The large nubs 75, 85 also facilitate cleaning, as opposed to prior art designs with smaller gear teeth. Further, a back end 92 (FIG. 5) of the second section 22 may include an aperture to provide for entry and exit. The relative sizes of the interior 24 of the first section 21 and the drive shaft 42 are designed to allow water and steam to axially move along the length of the shaft 20. Further, bushings 45 located along the shaft include flow passages to allow movement of water and steam.

The driver 10 may be used for procedure involving the cervical spinal area. The adjustable angle α and reduced length of the second section 22 allows for access within the limited space available. Driver 10 may also be used for other surgical applications, and for accessing other regions of the spine, including the thoracic, lumbar and/or sacral portions of the spine.

FIG. 10 illustrates another embodiment with the second section 22 pivotally mounted to the first section 21. One or more pivot connections 97 provide for pivotally positioning the second section 22 relative to the first section 21. A locking device such as but not limited to a set screw or ball-and-socket arrangement may be provided to lock the angular positions.

The term “distal” is generally defined as in the direction of the patient, or away from a user of a device. Conversely, “proximal” generally means away from the patient, or toward the user. Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. A surgical driver comprising:
a first shaft;
a second shaft;
a mount operatively connected to the second shaft and including an opening to receive tool bits; and
a drive mechanism extending through the first and second shafts to provide rotational power to the mount, the drive mechanism including a first wheel positioned in the first shaft and a second wheel positioned in the second shaft, each of the wheels including a pattern of adjacent nubs and valleys, each of the nubs includes an outer rounded end;
the first and second shafts meshing together with the nubs of the first wheel meshing with the valleys of the second wheel.

2. The driver of claim 1, wherein the second shaft is positioned at a non-coaxial orientation relative to the first shaft.

3. The driver of claim 1, wherein a length of the second shaft is smaller than the first shaft.

4. The driver of claim 1, wherein the second shaft is pivotally connected to the first shaft.

5. The driver of claim 1, wherein a distal end of the first shaft and the second shaft are formed as a single unit.

6. The driver of claim 1, wherein the first wheel includes a gear member with a plurality of outwardly-extending spaced-apart arms and the second wheel includes an open central area with the nubs being independent and spaced-apart.

7. The driver of claim 1, wherein the mount includes a head with an opening sized to receive the tool bits, a clip movably positioned within the head, and a locking ring rotatably mounted to the head, the locking ring rotatable between first and second positions to radially move the clip relative to the opening between locked and unlocked orientations.

8. The driver of claim 1, wherein the first and second shafts are hollow and a proximal end of the first shaft includes an opening to introduce a cleaning material into the first and second shafts.

9. A surgical driver comprising:
a first shaft;
a second shaft operatively connected to and positioned at a non-coaxial angle relative to the first shaft;
a mount operatively connected to the second shaft and configured to receive tool bits; and
a drive mechanism that extends through the first and second shafts to provide rotational power to the mount, the drive mechanism including a first wheel positioned at a distal section of the first shaft and a second wheel positioned at a proximal section of the second shaft, each of the wheels including a body with a plurality of spaced-apart nubs, each of the nubs including rounded axial and lateral sides;
the nubs of the first and second wheels meshing together to transfer the rotational power from the first shaft to the second shaft to rotate the mount.

10. The driver of claim 9, wherein one of the first and second wheels includes a gear member with outwardly-extending spaced-apart arms and the nubs positioned at ends of the arms.

11. The driver of claim 10, wherein the nubs of the other of the first and second wheels are independent and extend axially outward from the body and are spaced apart by the valleys.

12. The driver of claim 9, wherein the first shaft includes ridges that extend axially along the outer edge and align with the nubs.

13. The driver of claim 9, wherein the second shaft and the distal section of the first shaft are constructed as a unitary member.

14. The driver of claim 13, wherein the first wheel and the second wheel are positioned within the unitary member.

15. A surgical driver comprising:
a hollow shaft including a distal section with a distal end and a proximal section with a proximal end;
a mount attached to the distal end, the mount including an adjustable opening sized to receive one of a plurality of tool bits; and
a drive mechanism positioned within an interior of the shaft and including a drive input at the proximal end of the shaft and a connection at the distal end of the shaft to connect to the mount, the drive mechanism also including a connection mechanism to position the distal section at a non-coaxial orientation with the proximal section, the connection mechanism including a first wheel and a second wheel that mesh together and include nubs with rounded axial and lateral edges.

16. The driver of claim 15, wherein one of the first and second wheels includes a gear member with a plurality of arms that extend radially outward from a center of the wheel with a nub positioned at an end of each of the arms.

17. The driver of claim 16, wherein the nubs of the other of the first and second wheels are each independent and positioned around a central opening.

18. The driver of claim 15, wherein the opening of the mount is positioned within a collet and a movable clip is positioned within the collet, the movable clip radially movable within the collet from a first position towards a center of the opening to lock one of the plurality of tool bits within the opening and a second position away from the center of the opening to unlock the one tool bit.

19. The driver of claim 15, wherein the drive input includes a first end with a fitting and a second end with an aperture sized to receive the drive shaft, the drive input further including one or more vents that open into the hollow shaft.

20. The driver of claim 19, further including at least one bushing attached to the drive shaft, the bushing being positioned within the interior of the shaft and including spaced apart arms.

21. A method of using a surgical driver comprising:
rotating a proximal section of a drive mechanism that is positioned within a shaft of the surgical driver;
engaging the proximal section of the drive mechanism with a distal section of the drive mechanism by meshing together a first wheel connected to the proximal section with a second wheel connected to the distal sections; each of the first and second wheels including a plurality of rounded nubs positioned in a circular pattern and spaced apart by valleys, the first wheel and the second wheel positioned at a non-coaxial angle; and
rotating a mount connected to the distal section, the mount including an opening that holds a tool bit.

22. The method of claim 21, further comprising changing the non-coaxial angle formed by the first and second wheels.

23. The method of claim 21, further comprising introducing a cleaning material through openings in the proximal section and moving the cleaning material to clean the nubs on the first and second wheels.
